(19) United States
(12) Patent Application Publication
(10) Pub. No.: US 2008/0161818 A1
(43) Pub. Date: Jul. 3, 2008

(54) Spinous Process Distractor

(76) Inventors: Henning Kloss, Ennetburgen (CH); Bjorn Schafer, Ruppichteroth (DE)

Correspondence Address: J C PATENTS, INC., 4 VENTURE, SUITE 250, IRVINE, CA, 92618, US

(21) Appl. No.: 11/815,757

(22) PCT Filed: Feb. 8, 2006
(86) PCT No.: PCT/DE2006/000212

§ 371 Date: Aug. 7, 2007

Related U.S. Application Data
(60) Provisional application No. 60/661,926, filed on Mar. 16, 2005.

(30) Foreign Application Priority Data
Feb. 8, 2005 (DE) 10 2005 005 694.6

(51) Int. Cl. A61B 17/58

(52) U.S. Cl. 606/90

(57) ABSTRACT

The present invention relates to a device for the distraction of the spinous processes of two adjacent vertebrae. The spinous processes of the two vertebrae are supported on two separated distraction means being attached to a central body which can be expanded in an infinitely variable manner. The central body can be moved apart in an infinitely variable manner and be fixated along at least one guiding means. By moving apart the central body, the distraction means are expanded along the axial axis and thus distract the spinous processes of the two vertebrae supported on them. Furthermore, holding means can provide for a stable positioning of the implant and prevent an unwanted movement of the device after the implantation.

The present invention relates to a device for the distraction of the spinous processes of two adjacent vertebrae. The spinous processes lie on distraction means which can be expanded in an infinitely variable manner. Both distraction means are mounted on a central body which can be expanded in an infinitely variable manner and wherein the degree of the expansion determines the distance between the distraction means and thus the degree of the distraction of the spinous processes to each other.

In the state of the art only embodiments for the distraction of spinous processes are known which do not allow for an infinitely variable expansion or distraction of the spinous processes of two adjacent vertebrae. The distraction is achieved by spacers of defined height.

Thus, U.S. 2003/0065330 A1 discloses a device for the distraction of the spinous processes of vertebrae, wherein two vertical fixation means are mounted on a horizontal bar. The spinous processes of the vertebrae lie on a spacer which can be opened up by means of the horizontal bar and the thickness of which determines the degree of the distraction. Subsequent to the implantation, the degree of the distraction can no longer be modified.

A device allowing for an infinitely variable expansion, but not for a linear expansion is disclosed in U.S. Pat. No. 6,733,534 B2. Said patent describes an elastic hollow body made of a polymeric material which body can be filled and which is placed between two adjacent vertebrae and is subsequently widened by means of filling with a biological material until it has obtained a certain thickness.

The present invention aims at providing a device for the distraction of the spinous processes of two adjacent vertebrae which allows for an infinitely variable adjustment of the degree of distraction during the implantation.

This aim is realized by providing a device for the distraction of the spinous processes of two adjacent vertebrae, comprising a central body having two distraction means, where the two distraction means can be expanded in an infinitely variable manner only along the axial axis by a translational movement and be fixated there. Further advantageous designs, aspects and details of the invention result from the dependent claims, the description, the examples and the figures.

The present invention relates to a device for the distraction of the spinous processes of two adjacent vertebrae, comprising a central body 1 with at least two distraction means 2A and 2B, wherein the at least two distraction means 2 can be expanded in an infinitely variable manner. Preferably, there are two or four distraction means, i.e. one or two distraction means per spinous process. Particularly preferred are two distraction means 2A and 2B, hence only one distraction means per spinous process. The part of the device which the spinous processes principally lie on is indicated as distraction means 2. According to the invention, at least two distraction means 2 will be required, which means one distraction means 2 per spinous process on which distraction means 2 the corresponding spinous process is supported. By spreading the two distraction means 2A, 2B, i.e. by moving apart the two distraction means 2A and 2B along the axial axis, the distraction of the spinous processes is effected and thus also the distraction of the two vertebrae to be treated. Therefore, according to the invention, both distraction means are expandable in an infinitely variable manner along the axial axis, i.e. the longitudinal axis running through the vertebral column. The implant is expanded exclusively along the axial axis. The size of the implant is not modified, neither in lateral, nor in ventral and dorsal directions. The axis through the back and abdomen is the ventral-dorsal axis. The axial axis is situated normal to said ventral-dorsal axis along the vertebral column and again perpendicular to the axial axis, the lateral axis runs laterally through the body.

The distraction of the distraction means is carried out in an infinitely variable manner exclusively along the axis, hence the implant modifies its dimension only along said axis. Thus, the expansion of the distraction means takes place along a straight line and not along a curved line. Besides, the implant according to the invention is not provided with any flexible components showing a certain resetting capacity due to the pressure of the spinous processes or yielding to the pressure of the spinous processes thus reducing the distraction distance again.

Linear expansion means that a certain distance covered by an external means or a means associated with the implant for expanding the distraction means leads to a defined expansion of the distraction means. If, for example, an external means is used for expanding the distraction means, a shifting of said means by 1 mm leads to an expansion of the distraction means of e.g. 1 mm. Any additional movement of said means leads to an additional expansion of the distraction means by 1 mm. If, for example, the fixation means having a conical tip described herein is used for expanding the distraction means, a 360° turn of the fixation means leads to an expansion of 1.8 mm. Any additional turn by 360° leads to an additional expansion of 1.8 mm respectively. Such a type of expansion is called infinitely variable.

By contrast, the expanding method described in U.S. Pat. No. 6,733,534 B2 also leads to an infinitely variable expansion but not to a linear expansion, since for example the filling with a first ml of biological material results in an expansion of 4 mm but the filling with a fourth ml of biological material only results in an expansion of e.g. 1.2 mm. One inconvenience of a non linear expansion consists in the fact that a surgeon can only adjust the pressure in a very imprecise way. Besides, elastic implants as described for example in U.S. Pat. No. 6,733,534 B2 are problematic since the implant can be deformed subsequent to the implantation due to its elasticity and the desired optimal distraction is lost again.

According to the invention, said distraction, i.e. the movement of the two distraction means 2A and 2B along the axial axis is effected in an infinitely variable manner. No pre-manufactured spacers with pre-defined height are placed between the distraction means, instead the distraction means 2A and 2B can be brought, in an infinitely variable way and in a linear way, in any distance to each other between a minimum distance and a maximum distance. The minimum distance is determined by the diameter of both distraction means 2A and 2B which are lying firmly on each other and is of about 5 mm. The infinitely variable range of the device according to the invention, i.e. the distance between the two distraction means 2A and 2B, is of 5-25 mm, preferably of 6.5-20 mm and in particular preferably of 8-16 mm.

According to the invention, the infinitely variable movement of the two distraction means 2A and 2B relative to each other along the axial axis is achieved by each distraction means 2 being attached on a guiding means 3 and the guiding means 3 being fixated by a central body 1, though being supported movably in axial direction. In this context, both distraction means can be arranged on the same guiding means 3.

Subsequent to the distraction and fixation of the distraction means, the chosen distraction distance remains unmodified. Once the implant has been permanently fixated, there are no more variations in size in axial direction, particularly no variations caused by the implant itself.

In the preferred embodiment with two distraction means 2A and 2B, the distraction means 2A is attached to the guiding means 3A and the distraction means 2B is attached to the guiding means 3B. Both guiding means 3A and 3B are supported on a central body 1 or in a central body such that at least one guiding means 3A or 3B and preferably both guiding means 3A and 3B can be displaced in direction of the axial axis relative to each other or can be distracted or respectively shifted or moved apart.

As guiding means 3A and 3B can be used for example guide bars, guide rods or guide tubes which are movably supported in or on the central body 1 along the axial axis in corresponding recesses or reception means. By a displacement of the guiding means 3, any distance between the distraction means 2A and 2B can be set. The guiding means 3 are fixated in their position by means of corresponding fixation means 4, thereby guaranteeing that the distance between the distraction means 2A and 2B is maintained permanently and the set distance, i.e. the degree of the distraction of the vertebrae is not reduced again by the pressure of the vertebrae processes acting on the distraction means.

In a preferred embodiment, it's not the distraction means 2A and 2B which are attached to movable guiding means 3A and 3B, which in turn are movably mounted on a one-piece central body 1, but instead central bodies composed of two pieces, three pieces or more pieces are used which can be expanded, stretched and/or shifted apart only along the axial axis.

Two-piece central bodies 1 are particularly preferred, wherein the distraction means 2A is attached to one piece of the central body 1A and the other piece of the central body 1B is attached to the distraction means 2B in a translationally stable manner. The term translationally stable attachment refers to a mounting fixated in one place which can allow for a rotational movement of the distraction means to be preformed, but no translational movement of distraction means 2A (or 2B, respectively) relative to the piece 1A (or 1B, respectively) of the central body can be carried out. The distraction means 2A and 2B are preferably mounted on the same side of the respective central body and less preferably on opposed sides.

In these preferred embodiments, the distraction means 2 are not mounted movably on the central body 1, but they are mounted such that they can not be moved on a piece 1A or respectively 1B of the central body and are moved apart by shifting apart the two pieces 1A and 1B of the central body 1.

The central body 1, preferably composed of two or more pieces, thus consists of two or more engaging, extendable pieces, which can be distracted, expanded, interleaved and/or which can slide along each other. In particular, central bodies 1 composed of two pieces 1A and 1B are preferred. The two distraction means 2A and 2B are situated at two different pieces of the central body and preferably on the same side of the central body.

At least one part 1A or 1B of the central body 1 is provided with at least one guiding means 3. If in a preferred embodiment only one guiding means 3 is used, said guiding means is preferably arranged in the center and furthermore it is preferred if it is not designed in form of a rod or tube, but in oval, triangular, quadrangular, polygonal form, in form of a stadium lane, in plano-convex or in star-like form, in order to impede a rotation of the parts of the central body with respect to each other. Correspondingly, the other piece 1B of the central body 1 is designed such that it can receive the at least one guiding means 3 of the other piece 1A. Along this at least one guiding means 3 the two pieces 1 A and 1 B of the central body can be moved apart in an infinitely variable way.

As guiding means 3 can be used for example the aforementioned guide bars, guide rods or guide tubes which are preferred when at least two guiding means 3A and 3B are used. In the initial state, both pieces 1A and 1B of the central body 1 are interleaved. In said position, both distraction means 2A and 2B also have the least distance between each other.

On each piece 1A or 1B of the central body 1, respectively, one distraction means is laterally mounted, thus allowing for the two distraction means 2A and 2B to be shifted apart to the same degree as the two pieces 1A and 1B of the central body 1 in an infinitely variable way, only along the axial axis, i.e. they are expanded when the two pieces 1A and 1B of the central body 1 are shifted apart in an infinitely variable way.

It is preferred that a translational movement of the two distraction means 2A and 2B relative to each other can only be carried out in one dimension, i.e. along the axis running through the spine.

The distraction means 2A is mounted laterally on the one piece 1A of the central body 1. Preferably, the mounting is carried out such that the distraction means 2A can execute a rotational movement around an axis which is perpendicular to the axial axis. The rotational axis on which the distraction means 2A is situated is thus perpendicular to the axis along which the central body can be expanded. The distraction means 2A/2B are supported in a rotationally flexible way, preferably of up to 20° for being capable of adjusting to the anatomical conditions. This is important for avoiding load peaks and bone atrophy resulting therefrom in the area of the contact zones between the bones and the implant.

The distraction means 2B is mounted laterally on the other piece 1B of the central body and preferably it is arranged on the same side of the central body 1 as the distraction means 1A. Furthermore, concerning the distraction means 2B, it is also preferred if the distraction means 2B is supported on a rotational axis which is perpendicular to the axial axis. Moreover, it is preferred if the rotational axis through the distraction means 2B is parallel to the rotational axis through the distraction means 2A.

The at least two distraction means 2A and 2B can have any form. Preferably, the two distraction means are designed in a plano-convex way, i.e. they have the shape of a cylinder which has been cut along its longitudinal axis. In the initial state of the device, i.e. in the non-expanded or non-spread or non-extended state of the central body, the two plane surfaces of the two distraction means 2A and 2B are plane and adjacent to each other so that both distraction means together form a cylindrical form or an ellipsoid form.

It is particularly preferred if the at least two distraction means 2A and 2B cannot be deformed. It is also particularly preferred if the complete device is not deformable or ductile or elastic. That means that the device, in particular the central body 1 and the two distraction means 2A and 2B are made of a hard material, such as for example medical stainless steel, titanium or titanium alloys, tantalum, chrome, cobalt-chrome-alloys, vanadium, tungsten, molybdenum, plastics such as for example PEEK (polyetheretherketone) as well as fiber-reinforced plastics which are only insignificantly deformed by the pressure exercised by the spinous processes.

By using these hard materials, it is assured that the degree of the expansion of the distraction means corresponds as well to the degree of the distraction of the spinous processes. On the other hand, an elastic material would be deformed in a higher degree than in the case of a smaller degree of expansion due to the pressure of the spinous processes which increases in an unproportional manner, so that no linear correlation would exist between the distance of the two distraction means and the distance between the two spinous processes. However, it is possible to use materials which have a minimal intrinsic flexibility, such as for example PEEK or UHMWPE, in order to prevent a bone atrophy in the region of the contact zones.

Thus, the central body has a defined form which apart from the expansion is not modified during implantation. Furthermore, the central body does not consist of an elastic material, nor can it be filled or expanded by means of pressure generated inside the central body. The central body consist of a metallic and/or non-polymeric and/or non-deformable material and/or of material which can not be expanded under pressure.

Once the at least two distraction means 2A and 2B or the at least two pieces of the central body 1 have been shifted apart from the interleaved, non-expanded initial state, at least one fixation means 4 must guarantee for the set distance being maintained permanently, i.e. that the distraction means permanently maintain the distance between each other and will not yield to the pressure executed by the spinous processes.

Pins, bolts, clamps, rods or screws can be used as fixation means, wherein threaded pins and threaded screws are preferred. In the embodiments having a one-piece central body and two distraction means attached to guiding means, at least two fixation means 4A and 4B are required. In the embodiments having a one-piece central body and a fixated distraction means and a distraction means attached to a guiding means or a two-piece central body, one fixation means 4 is sufficient.

Furthermore, it is preferred if the at least one fixation means 4 has a conically tapered tip 5. Said conically tapered tip further has an angle of preferably 45 degrees. The tip 5 is centrally arranged.

It is particularly preferred if this at least one fixation means 4 does not only serve for the fixation of the distraction means 2A and 2B in the extended state, but also allows for a simultaneous adjustment of the distance between the two distraction means 2A and 2B by means of the at least one fixation means 4.

This can for example be achieved by a guiding means 3 having a bevelled surface 7 which abuts on the tip 5 of the at least one fixation means 4. By a height adjustment of the at least one fixation means 4 the conical tip 5 slides along the beveled surface 7 of a guiding means 3 in the direction of the tapering end of this bent surface 7, by means of which the translational movement of the fixation means 4 along the axis through the fixation means 4 leads to a translational movement of the guiding means 3 along the axis through the guiding means 3 and simultaneously fixates the position of the guiding means 3. Preferably, the wedge-shaped surface 7 tapers in the same angle as the conically tapered tip 5 of the fixation means 4. In particular, it is preferred if these two angles are 45 degrees.

If embodiments having two guiding means 3A and 3B with respectively one mounted distraction means 2 and a preferably one-piece central body 1 are provided, it is preferred to use two fixation means 4A and 4B for moving and fixating the two guiding means 3A and 3B, that means one fixation means 4 per guiding means 3.

In another preferred embodiment of the present invention a one-piece central body 1 is used, to which a distraction means 2A is attached in a translationally stable manner and which is only capable of executing rotational movements. The rotational movements can only be carried out around the longitudinal axis of the distraction means 2A. Translational movements of the distractions means 2A in relation to the central body cannot be executed. Furthermore, the central body is provided with a recess for the reception of at least one guiding means 3 or 3A and 3B or 3A, 3B and 3C, wherein the second distraction means 2B is attached to the at least one guiding means such that it can execute rotational movements around its longitudinal axis but cannot execute translational movements relatively to the at least one guiding means. The distraction means 2A and 2B can be moved apart in translational movements by shifting the at least one guiding means along the axial axis, i.e. along the longitudinal axis of the vertebral column in the recess in the central body 1 which was provided for said purpose, by means of which a distraction of the spinous processes of two adjacent vertebrae abutting on the distraction means is achieved. Once the desired distraction has been realized, the implant is fixated in its expanded position by means of at least one fixation means.

It is advantageous if the fixated expansion is permanently maintained in all embodiments of the distractor for spinous processes according to the invention which is described herein, since no elastic materials are used which would yield to the load after a certain period of time and would thus result in the desired distraction of the spinous processes being reduced again.

Other embodiments according to the invention preferably use two-piece central bodies, which can be distracted along at least one guiding means in the direction of the axial axis and preferably only in the direction of the axial axis. Respectively one distraction means is arranged at the lateral surface in the direction of the lateral axis at the respective part of the central body. In said inventive embodiments, the distraction is not carried out by means of a device integrated in the implant, such as the fixation means 4 having a conical tip 5, but by means of an external distraction means which is not implanted but only used temporarily during surgery.

An example for such a distraction means are distraction tongs which are applied in one recess at a piece of the central body 1A and 1B respectively. By means of such distraction tongs, the implant is expanded in axial direction, preferably in a linear and infinitely variable manner. Once the desired distraction has been achieved, both parts of the central body 1A and 1B are fixated relatively to each other by means of at least one fixation means. Said fixation means 4 can be a threaded screw having a flat or uneven or bevelled tip, which is screwed in, as shown in FIG. 4, until it fixedly abuts on a correspondingly bevelled surface of a guiding means or is pressed into a guiding means by means of pressure in order to thus fixate the central body. Therefore, the central body can be distracted and fixated in situ. The implant size is not measured beforehand. The distraction tongs serve for the reception of the implant, for the infinitely variable distraction of the implant and thus for the application of the implant in one step.

Subsequent to the fixation, the implant maintains its distracted form without yielding to the pressure of the spinous processes and without affecting the distraction distance once said distance has been set and fixated.

The central body 1A, 1B is situated laterally to the spinous processes. Furthermore, the implants according to the invention are provided with a chamfered corner (directly below 2A or respectively 2B) in order that more than two levels can be simultaneously supplied and that the implants have sufficient space to be arranged one above the other, if several implants are used on top of each other, i.e. if implants are also applied to adjacent pairs of vertebrae. The posterior curvation of the central body serves for a better adaptation to the anatomy.

The distraction means serve as abutting surfaces for the spinous processes and can preferably be rotated by +/−20° to dampen occurring load peaks and to provide for the formation of an abutting surface (contact surface between anatomy and implant) of as large dimensions as possible. Thus, in all embodiments according to the invention the distraction means are arranged on the central body in such manner that rotational movements can be executed around their won longitudinal axis. Furthermore, the distraction means are round, oval or plano-convex, such that the abutting surface for the spinous processes is of as large dimensions as possible.

In order to provide for a stable position of the implant, the spinous processes are framed between a holding means 6A, 6B and the respective part of the central body 1A or respectively 1B. In this context, the use of a spring element is preferred, so that the respective holding means automatically locks in place when being distracted. Thus, the position of both the holding means 6A and 6B in order to prevent a dislocation of the implant is assured. The holding means 6A and 6B are placed on the opposite side of the central body at the spinous processes and prevent the implant from being dislocated.

The holding means can be spread out from that side of the vertebral column which is opposite of the implant, but an additional surgical intervention would be required for said step. Thus, the two holding means are preferably spread out from the side opposite of the central body. For said purpose, a bore or respectively a recess is provided in the central body, by means of which a corresponding instrument can be inserted and reach the holding means, in order to spread out said holding means manually or by means of a spring mechanism which may be triggered by pressure.

Thus, an unilateral insertion (only one side of the spinal column is made accessible) of the implant according to the invention is made possible, thanks to which the intervention is smaller, the trauma for the patient is reduced and a shorter period of time is required for surgery.

In all the embodiments described herein the at least one guiding means is essentially parallel to the axial axis running through the vertebral column and seen along the longitudinal axis through the distraction means the two distraction means 2A and 2B are arranged in perpendicular position to the guiding means. It is furthermore preferred that the distraction means can move rotationally around their longitudinal axis, so that they can optimally adapt to the abutting spinous process. It is furthermore preferred that the at least one fixation means along its longitudinal axis is arranged in perpendicular position to the guiding means, wherein an arrangement in perpendicular position to the longitudinal axis of the distraction means is further preferred.

Thus, the device according to the invention for the distraction of the spinous processes of two adjacent vertebrae comprises a central body 1 having two distraction means 2A and 2B, wherein the longitudinal axis of the central body runs along the vertebral column. If the central body corresponds to the guiding means or if the at least one guiding means is arranged in the central body, it is also arranged in parallel position to the axis through the vertebral column. The two distraction means 2A and 2B are arranged in perpendicular position with respect to the axis running through the vertebral column and serve for distracting the spinous processes along the axis running through the vertebral column. In order to securely lock the spinous processes, each distraction means is provided with a holding means 6A or respectively 6B at the extremity opposing the central body. Distraction means and holding means are arranged such that rotational movements around the longitudinal axis running through the respective distraction means can be carried out. Furthermore, at least one fixation means serves for the fixation of the implant in its distracted position or, additionally to the fixation, it may also be capable of effectuating the infinitely variable distraction. The fixation is permanent and the distraction or respectively the distraction distance is not modified by the force exercised by the spinous processes and can only be modified by the surgeon. The distraction or respectively distraction distance refers to the distance by which the implant is distracted, i.e. expanded.

Other preferred embodiments of the present invention further are provided with at least one holding means 6 and preferably with one holding means 6 per distraction means 2. Preferably, the holding means 6A is fixated at the side of the distraction means 2A which is not facing the central body and the holding means 6B is fixated at the side of the distraction means 2B which is not facing the central body.

The preferably two holding means 6A and 6B are designed in the form of a sickle or in the form of a half moon; they are plane and arranged such that on one end they can be rotated around an axis running through the respective distraction means. By means of such an arrangement, the holding means 6 can be provided in a retracted initial state and in a spread out state. In the retracted initial state, the two holding means 6A and 6B are lying on each other like two sickles, wherein the center of rotation of the holding means 6A forms the center of the holding means 6B and the center of rotation of the holding means 6B forms the center of the holding means 6A.

When the central body is expanded or respectively when the distraction means are moved apart, this results in both holding means 6A and 6B spreading out at least partially. The at least one holding means 6 provides fixation of the device subsequent to the implantation between the spinous processes resting on it, so that an undesired sliding down, sliding away, slipping or shifting of the device between the vertebrae will be avoided.

In addition to said possibility of spreading out the preferably two holding means 6A and 6B, other possibilities may also be realized. Another possibility consists in spreading out a holding means by means of a spring mechanism which is triggered by contacting a certain spot at the holding means or at the distraction means. Further preferred, the holding means may be spread out by means of an external device which is preferably inserted through a corresponding bore in the central body from the side opposite of the distraction means and pushed in until it reaches the respective holding means. Said bore can be provided in the central body, but it may also be continued in the distraction means. Furthermore it is preferred if the spreading out of the holding means is reversible, such that a potential reimplantation can be carried out without any problems being caused.

For an easier insertion into the foramen interspinosus, a tapered cover 8A or respectively 8B can be provided on or above the holding means 6A and 6B. In FIG. 3, these two covers which approximately have the form of a quarter of a sphere are displayed. Said design, however, is not obligatory. However, covers 8A and 8B which should taper in a certain manner, are advantageous. Furthermore, it is preferred if both covers 8A and 8B have a perimeter similar to that of the corresponding distraction means 2A and 2B. These covers 8A and 8B can be arranged in central position on the holding means 6A and 6B; nonetheless an offset arrangement of the covers 8A or 8B is preferred, since this also contributes to an easier insertion into the foramen interspinosus. Instead of an offset or decentered arrangement of the covers 8A or 8B it is also possible to use covers with decentered or respectively offset tip. Both covers 8A and 8B are preferably supported in such way that a rotation around the axis running through the respective distraction means 2 can be carried out.

Furthermore, it is preferred if the individual components of the device according to the invention are coated with a ceramic coat. Ceramic coats comprise nitrides, carbides, phosphides preferably from semi-metals and metals or respectively metal alloys. Examples for ceramic coats are boron nitrides, titanium niobium nitride, titanium calcium phosphide (Ti—Ca—P), Cr—Al—N, Ti—Al—N, Cr—N, TiAlN—CrN, Ti—Al—C, Cr—C, TiAlC—CrC, Zr—Hf—N, Ti—Hf—C—N, Si—C—N—Ti, Si—C—N as well as DLC (Diamond Like Carbon).

### LIST OF ABBREVIATIONS

1 central body
1 A piece A of the central body
1 B piece B of the central body
2 distraction means
2A distraction means A
2B distraction means B
3 guiding means
3A guiding means A
3B guiding means B
3C guiding means C
4 fixation means
4A fixation means A
4B fixation means B
5 conical tip of the fixation means
6 holding means
6A holding means A
6B holding means B
7 surface abutting on the tip of the fixation means
8A cover A
8B cover B
9A/B recess for the application of distraction tongs
10A snap action mechanism for holding means 6A
10B snap action mechanism for holding means 6B

### DESCRIPTION OF THE FIGURES

FIG. 1 shows a side view of an embodiment of the device according to the invention in partially interleaved initial state with spreading out holding means 6A, 6B in the form of a sickle;

FIG. 2 shows a side view of an embodiment of the device according to the invention with a two-piece central body 1 in the interleaved initial state and the two distraction means 2A, 2B abutting with their plane surfaces and the two holding means 6A, 6B having the form of a sickle in the retracted state;

FIG. 3 shows a side view of an embodiment of the device according to the invention along an axis running trough the distraction means 2 having a two-piece central body 1A, 1B in the expanded state and with fixation means 4 which can be screwed in, distraction means 2A, 2B which are expended and holding means 6A, 6B which are in spread out state;

FIG. 4 shows a side view of an embodiment of the device according to the invention from the side opposite of the distraction means 2. A two-piece central body 1A, 1B in partially expanded state and with fixation means 4 which are partially screwed in, can be seen, wherein the conically tip 5 of the fixation means 4 abuts on the correspondingly bevelled surface 7 of the piece of the central body 1 which is not provided with the fixation means 4.

FIG. 5 shows the expanded form of another embodiment of the device according to the invention, having a two-piece central body 1A, 1B and a guiding means 3 in form of a stadium lane, the two distraction means 2A and 2B as well as the two holding means 6A and 6B which may for example be spread out by means of a snap action mechanism 10A or respectively 10B;

FIG. 6 shows a side view of the implant according to FIG. 5 in non-distracted state;

FIG. 7 shows another side view of the implant according to FIG. 5 in compressed state;

FIG. 8 shows a schematic view of a further embodiment of the device according to the invention in its simplest version;

FIG. 9 shows a further perspective view of the embodiment according to FIG. 8, wherein a rod-like guiding means 3 as well as two distraction means which can be moved along the guiding means are schematically displayed;

FIG. 10 shows another view of the embodiment according to FIG. 8 in ventral direction (i.e. in direction of the abdomen);

FIG. 11 shows another display of the embodiment according to FIG. 8 in lateral direction.

### EXAMPLES OF EMBODIMENTS

Preferred embodiments of the device according to the invention will now be discussed on the basis of the examples, wherein it is to be understood that the discussed examples show advantageous embodiments of the invention, but that the scope of the invention is not limited to these embodiments.

### Example 1

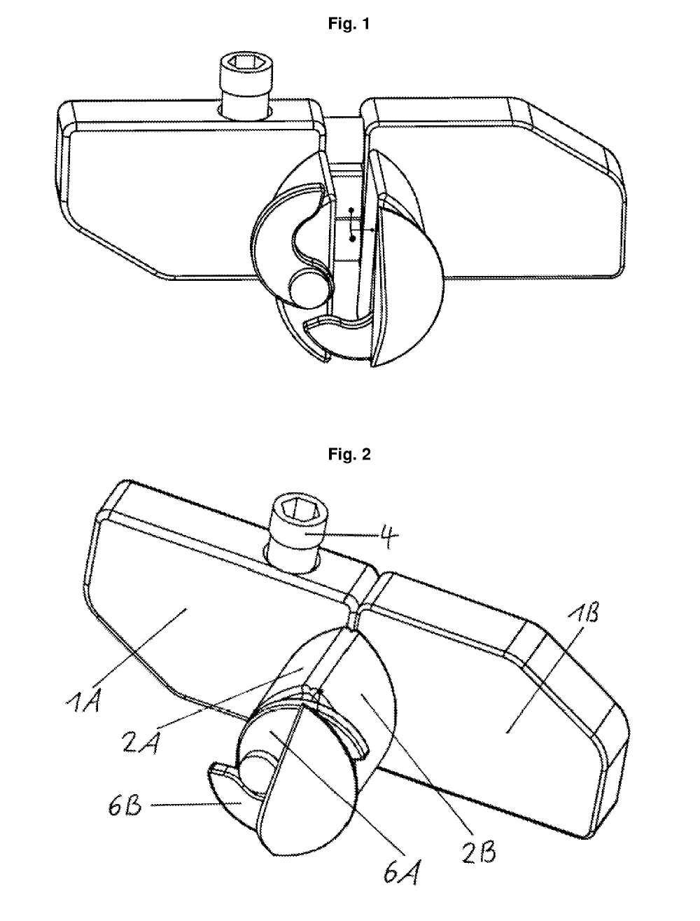

FIG. 1 shows a preferred embodiment the individual components of which are made of titanium and comprise a two-piece central body 1A and 1B, three guiding means 3A, 3B, 3C, two distraction means 2A and 2B, one fixation means 4 as well as two holding means 6A and 6B.

FIG. 2 shows one embodiment of the device according to the invention in the non-expanded, interleaved initial state. The two pieces of the central body 1A and 1B abut on each other. Also, the plane surfaces of the two plano-convex distraction means 2A and 2B lie upon each other so that both distraction means 2A and 2B together form a cylinder or a compressed cylinder. The fixation means 4 in the form of a threaded screw is screwed out so that one part of the threaded screw projects out of the one piece 1A of the central body 1. The two holding means 6A and 6B having the form of a sickle and being arranged on the head of the distraction means 2 are in retracted state.

The device is implanted in its initial state, i.e. it is inserted between the two spinous processes of the vertebrae which are to be distracted, without a transection of the ligament supraspinosus being required, which is a great advantage provided by the implants according to the invention. Subsequent to being placed between the spinous processes, the device is opened out or expanded by screwing in the threaded screw.

FIG. 4 shows a view of the two-piece central body 1 from the side opposite of the two distraction means 2. Three guiding means are provided, wherein two of them are designed as pins in the form of cylinders. The two pins 3A and 3C in the form of cylinders are either attached to piece 1A or to piece 1B of the central body or are actually part of the central body itself or one guiding means 3A or 3C is arranged on the piece 1A and the other guiding means on the piece 1B or they belong to the pieces 1A or 1B, respectively. The other piece of the central body has corresponding bores or recesses for the reception of the guiding means 3A and 3C.

The piece 1B of the central body 1 which is not provided with the fixation means 4 has a guiding means 3B which on its free end tapers in an inclined surface 7 which abuts on the conical tip 5 of the fixation means 4.

The fixation means 4 designed as a threaded screw can be screwed into the piece 1A of the central body 1 along an axis which is perpendicular to the axial axis, i.e. the axis along the vertebral column and perpendicular to an axis which runs through a distraction means 2.

The fixation means 4 has a tapering end 5. The conical end 5 preferably has an angle of 45°. The bent surface 7 of the guiding means 3B also preferably has an angle of 45°, so that the surface 7 and tip 5 meet each other along a straight line.

When the fixation means is screwed into the piece 1A of the central body 1 along its central axis, the straight line, on which surface 7 and tip 5 meet, moves in direction of the edge of surface 7, thus initializing a translational movement of both pieces 1A and 1B of the central body 1 which move apart along the axial axis. At the same time, the fixation means 4 prevents both pieces 1A and 1B of the central body 1 from being interleaved again due to the pressure exercised by both spinous processes. The guiding means 3A and 3C guarantee that the translational movement of the two pieces 1A and 1B of the central body 1 can only be effected in axial direction and that no additional rotational movement of the two pieces 1A and 1B around each other is carried out simultaneously.

In the case of maximum expansion of the central body 1, the fixation means 4 is completely screwed into the piece 1A of the central body and the outer tip of the conical tip 5 has reached the outer edge of the inclined surface 7. The two pieces 1A and 1B of the central body 1 are then maximally expanded and the expansion is of 20 mm.

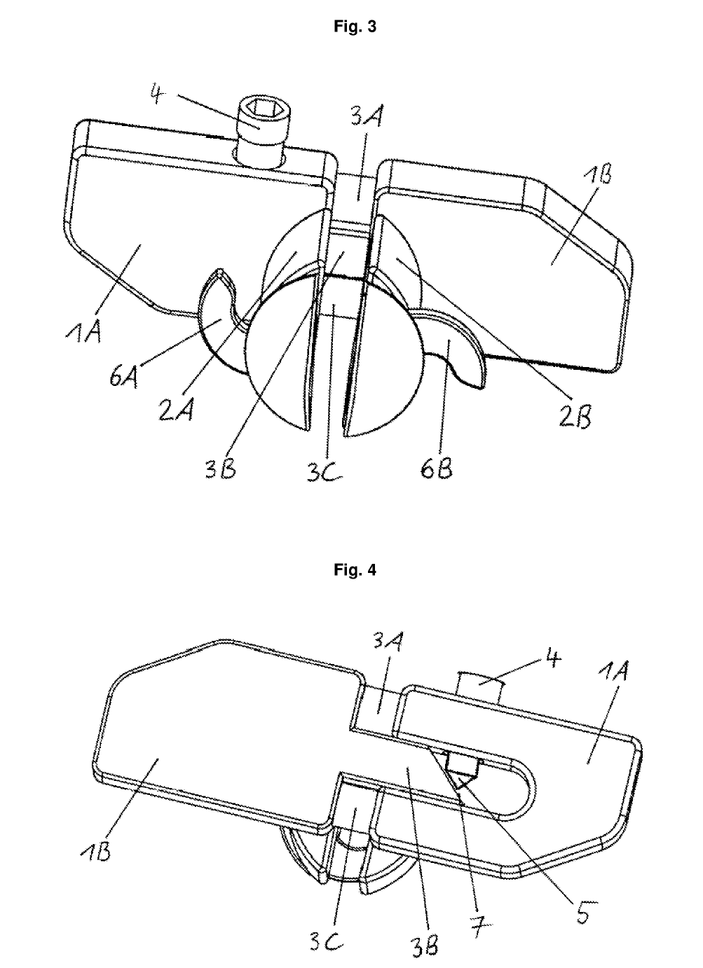

A front view of the device in the expanded state is provided by FIG. 3. The two pieces 1A and 1B of the central body 1 are shifted apart along the axes running through the two guiding means 3A and 3C. The distance between the two pieces 1A and 1B of the central body to each other now is the same distance as that of the two distraction means 2A and 2B to each other. The fixation means 4, except for the screw head, is screwed into the piece 1A of the central body. The two holding means 6A and 6B on the outer surfaces of the distraction means 2A and 2B are spread out and guarantee for a firm position of the device between the vertebral bodies as they lock the corresponding spinous process which is situated on the corresponding distraction means between themselves and the corresponding piece of the central body 1. Thus, the spinous process situated on the distraction means 2B is fixated between the holding means 6B and the piece 2B of the central body 1 with the help of the holding means 6B.

### Example 2

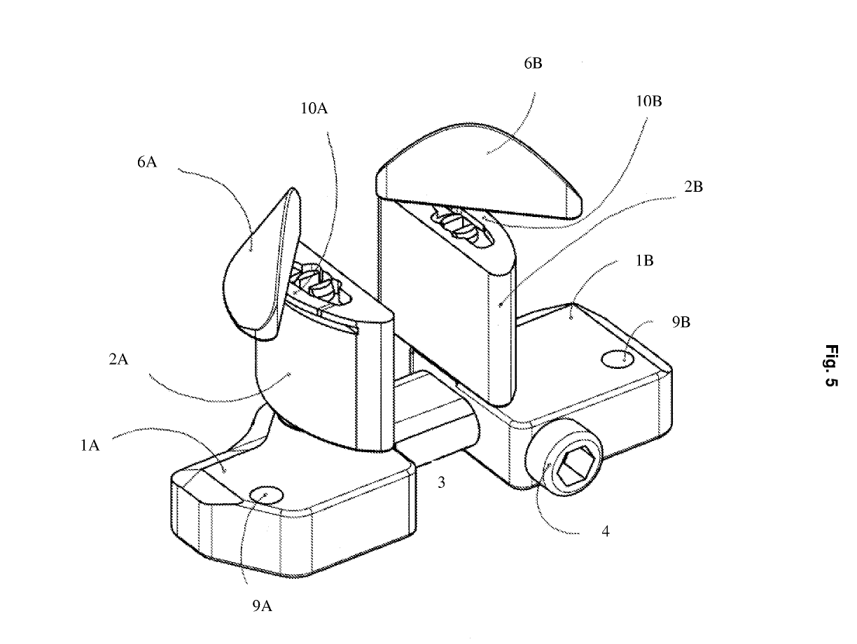

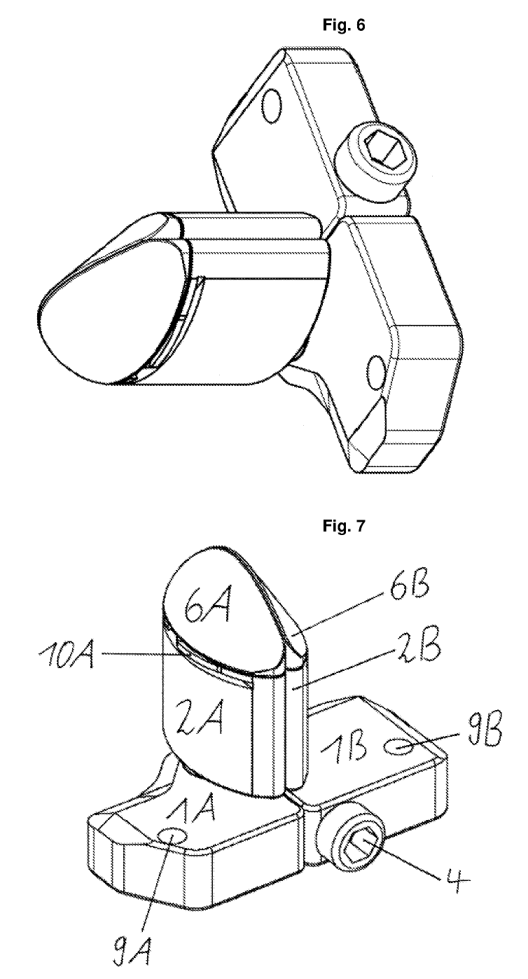

FIG. 5 shows another preferred embodiment of the present invention, the single components of which are made of titanium and comprise a two-piece central body 1A and 1B, a guiding means 3, two distraction means 2A and 2B, one fixation means 4 as well as two holding means 6A and 6B.

Each part of the central body 1A and 1B has a recess 9A or respectively 9B for the reception of distraction tongs.

FIG. 5 shows the central body in distracted state. The one guiding means 3 is designed as a circular path, like a running lane in a stadium, in order to prevent relative rotations of the two pieces of the central body 1A and 1B to each other. The guiding means 3 is not fixedly connected to the piece 1A of the central body, nor can it be moved relatively to the piece 1A; or it may form an entity with the piece 1A of the central body. Piece 1B of the central body is provided with a corresponding recess for the reception of the guiding means in a translational movement, so that both parts of the central body can be expanded or respectively distracted by means of a sliding movement of the guiding means in the recess in piece 1B in an infinitely variable linear manner along the axial axis.

Once the desired distraction has been realized, the two pieces of the central body 1A and 1B are fixated in relation to each other with the help of the fixation means 4, in this case designed in form of a threaded screw. Due to the exertion of pressure on the fixation means the distraction distance is fixated by means of the fixation means.

The two holding means 6A and 6B are spread out by means of the respective snap action mechanism 10A or respectively 10B and lock the respective spinous process between themselves and the opposed piece 1A or respectively 1B of the central body.

The device according to the invention is implanted in its initial state as shown in FIG. 6, i.e. it is inserted between the two spinous processes of the vertebrae to be distracted without a transection of the ligament supraspinosus being required, which is a great advantage of the implants according to the invention. The central body is placed laterally to the vertebral column, i.e. it is situated on the plane created by the axial axis and the ventral-dorsal axis. Subsequent to the insertion between the spinous processes, the device is expanded or respectively distracted by means of external distraction tongs.

For said purpose, the distraction tongs are inserted in the recesses 9A and 9B and the implant is expanded in infinitely variable manner by said distraction tongs until the desired distraction is achieved. The fixation means in the form of a threaded screw is tightened until a fixation of the distracted pieces 1A and 1B of the central body to each other is achieved by means of exerting corresponding pressure on the fixation means.

With the help of another external tool or with the help of a snap mechanism provided on the implant and triggered by an external tool, the holding means are spread out by means of direct contact in order to fixate the whole implant. Said external tool is introduced from the opposite side of the central body through a corresponding guidance in the central body traversing the central body in order to trigger the snap action mechanism. Said embodiment has the additional advantage that only one side of the vertebral column has to be accessible during the implantation process and that the implant can be inserted between the adjacent spinous processes from that side and that also the holding means can be spread out from that side.

### Example 3

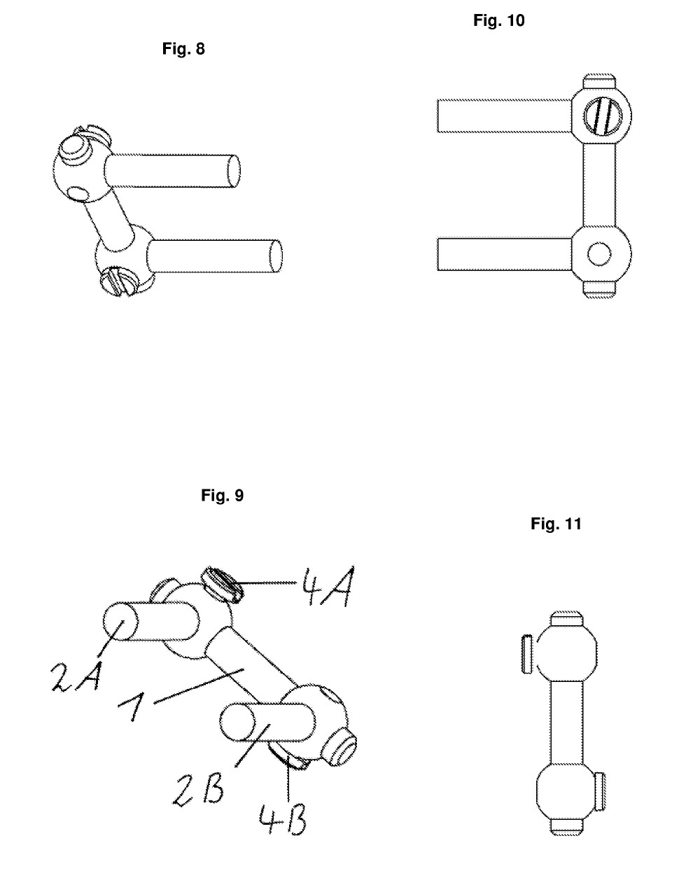

FIGS. 8 to 10 show the simplest embodiment, in which the central body 1 is designed in one piece and two distraction means 2A and 2B can be expanded along the central body and along the axial axis in an infinitely variable manner by means of external distraction tongs.

Both distraction means are provided with a fixation means 4A and 4B for fixating the distraction means relatively to each other. The two fixation means are designed in form of a threaded screw. In FIGS. 8-10, the distraction means 2A and 2B are only indicated schematically in form of tubes and the holding means are not displayed.

1. Device for the distraction of the spinous processes of two adjacent vertebrae, comprising a central body (1) having two distraction means (2A, 2B), characterized in that said two distraction means (2A, 2B) can be expanded in an infinitely variable manner only along the axial axis by a translational movement and be fixated there.

2. Device according to claim 1, characterized in that the central body (1) is dimensionally stable and not ductile or deformable.

3. Device according to claim 1, characterized in that the central body (1) is composed of two pieces.

4. Device according to claim 3, characterized in that the distraction means are dimensionally stable and not deformable.

5. Device according to claim 3, characterized in that the two-piece central body (1) consists of two engaging, extendable pieces (1A, 1B) which can be expanded, interleaved and/or can slide along each other.

6. Device according to claim 3, characterized in that at least one piece of the central body (1) is provided with at least one guiding means (3) and that the other piece of the central body (1) is designed such that it can receive the at least one guiding means (3).

7. Device according to claim 6, characterized in that the central body (1) can be expanded in an infinitely variable manner along the longitudinal axis of the at least one guiding means (3).

8. Device according to claim 1, characterized in that the distraction means (2A, 2B) can be moved apart in an infinitely variable manner and in a linear manner along the axial axis.

9. Device according to claim 3, characterized in that one distraction means (2A, 2B) is laterally arranged on both pieces of the central body (1A, 1B).

10. Device according to claim 1, characterized in that the distraction means (2A, 2B) are designed in a plano-convex manner.

11. Device according to claim 1, characterized in that the distraction means (2A, 2B) are respectively arranged such that they can be rotated around an axis which is perpendicular to the axial axis.

12. Device according to claim 1, characterized in that the expendable central body (1) has at least one fixation means (4).

13. Device according to claim 12, characterized in that the at least one fixation means (4) is a threaded pin or a threaded screw.

14. Device according to claim 12, characterized in that the at least one fixation means (4) has a conically tapered tip.

15. Device according to claim 14, characterized in that the piece of the central body (1) which is not provided with the fixation means (4) has a surface (7) which abuts on the conical tip (5) of the at least one fixation means (4).

16. Device according to claim 15, characterized in that the angle in which the conical tip (5) of the at least one fixation means (4) tapers, corresponds to the angle in which the surface (7) of one piece of the central body (1) abutting the conical tip (5) of the at least one fixation means (4) is bevelled.

17. Device according to claim 1, characterized in that the distraction means (2A, 2B) have at least one holding means (6A, 6B) on the side which is not facing the central body.

18. Device according to claim 1, characterized in that the at least one holding means (6) per distraction means (2) is arranged such that it can be spread out.

19. Device according to claim 1, characterized in that the at least one holding means (6) per distraction means (2) is designed in the form of a sickle.

20. Device according to claim 17, characterized in that the respective holding means (6) is arranged such that it can be rotated around an axis which is perpendicular to the axial axis and which is running through the respective distraction means (2).

21. Device according to claim 18, characterized in that the at least two holding means (6A, 6B) are arranged such that they are spread out simultaneously to the expansion of the distraction means (2A, 2B).
